SEQUENCE LISTING (13 sequences)

SEQ ID NO: 1 - skipped

SEQ ID NO: 2 - protein, 4 aa, artificial
  Protease cleavage sequence
GFLG

SEQ ID NO: 3 - protein, 4 aa, artificial
  Protease cleavage sequence
AFKK

SEQ ID NO: 4-5 - skipped (2 entries)

SEQ ID NO: 6 - protein, 4 aa, artificial
  Protease cleavage sequence
AFKK

SEQ ID NO: 7 - skipped

SEQ ID NO: 8 - protein, 5 aa, artificial
  Protease cleavage sequence
GGGRR

SEQ ID NO: 9 - protein, 6 aa, artificial
  Protease cleavage sequence
GILGVP

SEQ ID NO: 10 - protein, 8 aa, artificial
  Protease cleavage sequence
GPLGIAGQ

SEQ ID NO: 11 - protein, 7 aa, artificial
  Protease cleavage sequence
  Citrulline (3)..(3)
  homophenylalanine (5)..(5)
EPXGXYL

SEQ ID NO: 12 - protein, 5 aa, artificial
  Protease cleavage sequence
DEVDP

SEQ ID NO: 13 - protein, 8 aa, artificial
  Protease cleavage sequence
KGSGDVEG

(19) United States
(12) Patent Application Publication
(10) Pub. No.: US 2023/0285456 A1
(43) Pub. Date: Sep. 14, 2023

(54) TUMOR THERAPY COMPOSITIONS AND METHODS

(71) Applicants: Fu-Sen Liang, Albuquerque, NM (US); Wei Wang, Albuquerque, NM (US); Judy Cannon, Albuquerque, NM (US); Alex Huang, Cleveland, OH (US); Huong Nguyen, Cleveland, OH (US); UNM RAINFOREST INNOVATIONS, Albuquerque, NM (US); CASE WESTERN RESERVE UNIVERSITY, Cleveland, OH (US)

(72) Inventors: Fu-Sen Liang, Albuquerque, NM (US); Wei Wang, Albuquerque, NM (US); Judy Cannon, Albuquerque, NM (US); Alex Huang, Cleveland, OH (US); Huong Nguyen, Cleveland, OH (US)

(21) Appl. No.: 17/625,896

(22) PCT Filed: Jul. 17, 2020
(86) PCT No.: PCT/US2020/042508

§ 371 Date: Jan. 10, 2022

Related U.S. Application Data
(60) Provisional application No. 62/876,052, filed on Jul. 19, 2019.

(51) Int. Cl. A61K 35/17; C12N 15/62; A61P 35/00; A61K 47/66; A61K 38/17

(52) U.S. Cl. CPC A61K 35/17; A61K 38/177; A61K 47/66; A61P 35/00; C12N 15/62

(57) ABSTRACT

A modified T cell includes exogenous polynucleotides that encode components of a therapeutic expression system. A first exogenous polynucleotide encodes a therapeutic polypeptide operably linked to a regulatory region inducible by inducer. A second exogenous polynucleotide encodes polypeptide components of a chemical induced proximity (CIP) complex. A third exogenous polynucleotide that encodes a chimeric antigen receptor that specifically binds to an antigen.

### CROSS-REFERENCE TO RELATED APPLICATION

This application claims the benefit of U.S. Provisional Pat. Application No. 62/876,052, filed Jul. 19, 2019, which is incorporated herein by reference in its entirety.

### SEQUENCE LISTING

This application contains a Sequence Listing electronically submitted via EFS-Web to the United States Patent and Trademark Office as an ASCII text file entitled “0310-0116WO-01_ST25.txt” having a size of 4 kilobytes and created on Jul. 16, 2020. The information contained in the Sequence Listing is incorporated by reference herein.

### SUMMARY

This disclosure describes, in one aspect, a T cell that includes exogenous polynucleotides that encode components of a therapeutic expression system. A first exogenous polynucleotide encodes a therapeutic polypeptide operably linked to a regulatory region inducible by inducer. A second exogenous polynucleotide encodes polypeptide components of a chemical induced proximity (CIP) complex. A third exogenous polynucleotide that encodes a chimeric antigen receptor that specifically binds to an antigen.

In some embodiments, the regulatory region binds components of a chemical induced proximity (CIP) expression system.

In some embodiments, presence of the inducer in the T cell induces assembly of the CIP expression system and expression of the therapeutic polypeptide.

In some embodiments, the inducer can include abscisic acid (ABA), gibberellic acid (GA), or rapamycin.

In another aspect, this disclosure describes a method of treating a tumor in a subject. Generally, the method includes introducing into T cells exogenous polynucleotides to produce a modified T cell, administering the modified T cell to the subject, administering to the subject an inactive prodrug of the inducer, the inactive prodrug being activatable to the inducer by a tumor microenvironment-associated signal (e.g., a protease or hypoxia) within a solid tumor, allowing the inactive prodrug to be converted to the active inducer, and allowing the active inducer to enter the modified T cell and induce the CIP complex to express the therapeutic polypeptide. A first exogenous polynucleotide encodes a therapeutic polypeptide operably linked to a regulatory region inducible by inducer. A second exogenous polynucleotide encodes a chemical induced proximity (CIP) complex that is induced by an inducer molecule. A third exogenous polynucleotide that encodes a chimeric antigen receptor that specifically binds to an antigen expressed by cells of the tumor.

In some embodiments, the T cells are obtained from the subject.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Schematic design of cancer-triggered T cell immunotherapy.

FIG. 2. ABA prodrug for plasmin. (A) Synthesis and activation of ABA prodrug Plasmin-responsive Compound 1. Plasmin-responsive Compound 1 is cleaved by plasmin to generate ABA. (B) Stability of ABA prodrug and regeneration of ABA from Plasmin-responsive Compound 1 by plasmin analyzed using HPLC. 200 µM of Plasmin-responsive Compound 1 was incubated with plasmin in TRIS buffer (pH 7.4) at 37° C. for 30 minutes. Chromatogram showed cleavage of the prodrug comparing to ABA. (C) HEK293T reporter cells can respond to plasmin via Plasmin-responsive Compound 1, leading to EGFP expression. Reporter cells were treated with ABA or Plasmin-responsive Compound 1, with and without plasmin. Treated cells were incubated for 15 hours and EGFP expression was observed under fluorescence microscope.

FIG. 3. ABA prodrug for hypoxia. (A) Synthesis and activation of ABA prodrugs Hypoxia-responsive Compound 1 and Hypoxia-responsive Compound 2. Hypoxia-responsive Compound 1 and Hypoxia-responsive Compound 2 undergo 1-electron nitroreduction to generate functional ABA. (B) Stability of ABA prodrug and regeneration of ABA were analyzed using HPLC. 100 µM of Hypoxia-responsive Compound 1 and Hypoxia-responsive Compound 2 were incubated with NTR (50 mg/mL) and NADPH (1 mM) in PBS (pH 7.4) at 37° C. for 60 minutes and the cleavage were analyzed by HPLC. (C) EGFP expression of HEK293T-EGFP reporter cells with ABA, Hypoxia-responsive Compound 1, Hypoxia-responsive Compound 2, or Hypoxia-responsive Compound 1 and Hypoxia-responsive Compound 2 in the presence of NTR and NADPH. Cells were treated with indicated molecules and EGFP expression after 24 hours were observed under fluorescence microscope.

FIG. 4. ABA prodrug for β-galactosidase. (A) Synthesis and activation of ABA prodrug β-galactosidase-responsive (βGal-responsive) Compound 1. βGal-responsive Compound 1 is cleaved by β-galactosidase to generate ABA. (B) Stability of ABA prodrug and regeneration of ABA from βGal-responsive Compound 1 by β-galactosidase analyzed using HPLC. 200 µM of βGal-responsive Compound 1 was incubated with β-galactosidase in TRIS buffer (pH 7.4) at 37° C. for different time points. Chromatogram showed cleavage of the prodrug comparing to ABA. (C) HEK293T reporter cells can respond to β-galactosidase via βGal-responsive Compound 1, leading to EGFP expression. Reporter cells were treated with ABA, or βGal-responsive Compound 1, with and without β-galactosidase. Treated cells were incubated for 24 hours and EGFP expression was observed under fluorescence microscope.

FIG. 5. Cytotoxicity of Hypoxia-responsive ABA prodrugs and Plasmin-responsive ABA prodrugs. (A) CHO cells were treated with Hypoxia-responsive ABA prodrugs (or with DMSO as a control) at different concentrations. (B) CHO cells were treated with Plasmin-responsive ABA prodrug (or with DMSO as a control) at different concentrations. Cytotoxicity of ABA prodrugs was evaluated using MTT assay after 24 hours incubation. Viability % calculated by comparing to DMSO treatment. Error bars represent ± sem (n=3).

FIG. 6. Structure of esterase-resistant ABA prodrugs.

FIG. 7. Structure of rapamycin prodrugs for CAR plus T cells.

FIG. 8. Luciferase expression induced by ABA using pairs of ABI with different PYR1 mutants. Error bars represents ± s.e.m (n=3).

FIG. 9. Generalized retroviral DNA constructs for producing ABA-responsive CAR+ T cells. (A) General schematic design of an ABA-responsive split transcriptional activator. (B) General schematic design of an ABA-inducible reporter or ABA-inducible therapeutic protein. (C) Split CAR targeting CSPG4.

FIG. 10. Construction of split CAR and inducible gene expression system that is dependent on the presence of small molecule dimerizer. DNA constructs encoding a split CAR. (A) The first half includes a validated human HER2-recognizing scFv with the CD8 hinge region fused to CD8 transmembrane domain (CD8tm), 4-1BB costimulatory domain, and ABI. (B) The second half contains the ectodomain of DNAX-activating protein 10 (DAP10) linked to CD8tm, 4-1BB, PYR*(F61L/A160C), and CD3ζ immunoreceptor tyrosine-based activation motifs (ITAMs).

FIG. 11. A conventional, non-split version of CAR was constructed as a positive control for comparison of anti-tumor activity.

FIG. 12. Construction of split CAR and inducible gene expression system that is dependent on the presence of small molecule dimerizer. (A) The ABA-inducible gene cassette includes an ABA-dimerizable nuclear localized (by nuclear localization sequence, NLS) split transcriptional activator (P65/HSF1AD-PYR*-NLS and ZFHD1-ABI-NLS linked by a self-cleaving T2A peptide in plasmid. (B) A Z12-driven inducible therapeutic genes (expressing anti-PD-L1 and TRAIL linked by T2A in plasmid. ZFHD1 DNA binding domain (DBD) and P65 (aa 361-551)/HSF1 (aa 406-529) hybrid transcriptional activation domain are previously developed for gene therapy applications. The Z12 sequence contains 12 copies of consensus sequence recognized by ZFHD1-DBD.

FIG. 13. ABA-driven apoptosis. (A) ABA-driven secretion of apoptotic inducer TRAIL. HEK293T cells were transfected with the ABA split transcriptional activator (sv-vp-PYR*-ires-Gal4-ABI) and an inducible vector encoding secretable TRAIL (5xUAS-pminCMV-sTRAIL). Cells were then treated with ABA (1 µM) or DMSO as a control. The concentration of secreted TRAIL in the supernatant after 24 hours was determined via human ELISA kit. (B) sTRAIL-induced apoptosis in human breast cancer cell line. Cell culture supernatant containing sTRAIL was collected after 24 hours treated with either ABA (1 µM) or DMSO as a control, then incubated with TRAIL-sensitive MDA-MD-231 cancer cell lines for another 24 hours. Apoptosis cells were accessed using a dead cell apoptosis kit with Annexin V-FITC and PI, for flow cytometry. Recombinant human TRAIL (rhTRAIL) (6000 pg/mL) was used as a control. Error bars are ± sem (n=3), significant determined by Student’s t-test, **p ≤ 0.001.

### DETAILED DESCRIPTION OF ILLUSTRATIVE EMBODIMENTS

This disclosure describes effective and safe cancer immunotherapy for solid tumors that involve integrating a cancer-inducible prodrug strategy with CAR T cell therapy and immune checkpoint blockade. The therapy involves engineered T cells (e.g., CAR Plus T cells) that can be selectively triggered by the cancer microenvironment to provide locally boosted T cell activity and persistence as well as to produce anticancer agents in solid tumor environments.

Cancer immunotherapy harnesses a patient’s own immune system to combat cancers. Immune checkpoint inhibitors based on antibodies or small molecules block the immune checkpoint pathways by targeting regulators in the pathways (e.g., PD-1/PD-L1, CTLA4) to enhance the immune activity of patient’s effector T cells. Although immune checkpoint blockade can be effective against certain cancers, systemic administration of these inhibitors can cause severe immune-related adverse events since immune checkpoint pathways also are involved in regulating autoimmunity.

CAR T cell therapy engineers a patient’s T cells ex vivo to express tailored receptors that can more efficiently recognize specific antigens on cancer cells once transferred back to patients. This strategy has been demonstrated to be effective towards hematological malignancies (e.g., leukemia). Efficacy if CAR T cells in therapy for solid tumors remain challenging, however, due to limited T cell persistence and the immunosuppressive solid tumor microenvironment.

Although combined therapies including co-administration of immune checkpoint inhibitors or cytokines with CAR T cells can improve efficacy, systemic administration of these agents can cause serious toxicities. Furthermore, “on target off cancer” effects have been reported showing that CAR T cells can attack non-cancerous tissues if antigens targeted by CAR also are present on healthy tissues. The systemic enhancement of CAR T cells activity can exacerbate such issues. A new therapeutic strategy that allows targeted enhancement of T cell activity and persistence, and simultaneously produces extra anticancer agents only in tumor sites can offer a superior immunotherapy for solid tumors.

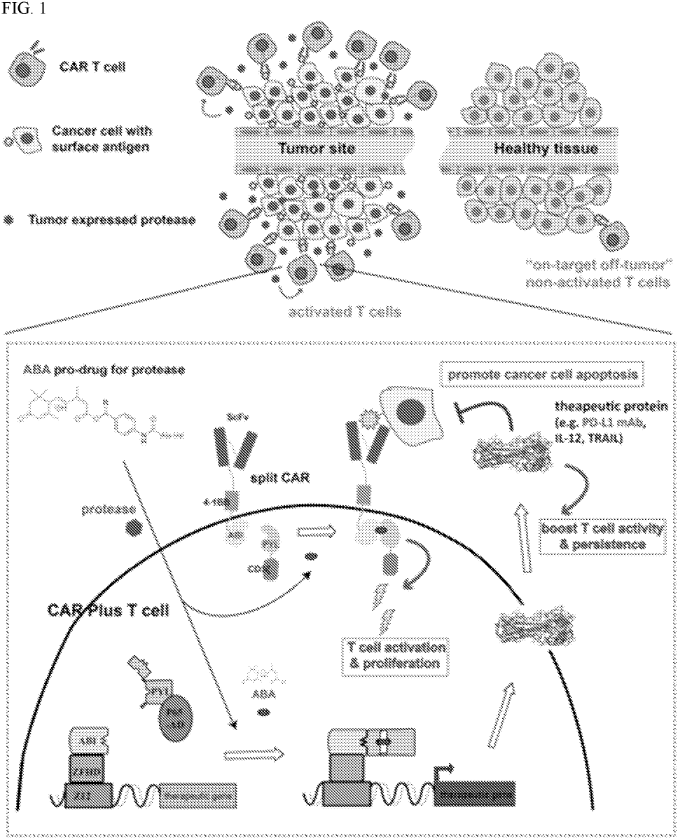

This disclosure describes a strategy for targeted stimulation of T cell activity and inducing more effective therapeutic effects only within solid tumor environments. The strategy involves a prodrug concept and a chemically-induced proximity (CIP) technology that can be applied to enable small molecule-induced gene expression (FIG. 1). The CIP technology uses a chemical inducer (i.e., abscisic acid, ABA) to trigger binding between unique inducer-binding adaptor proteins (i.e., PYL and ABI in the case of ABA) that can be genetically fused to any two proteins of interest (e.g., a DNA binding protein domain and a transcriptional activation protein domain for inducing gene expression). The ABA-induced interaction of engineered fusion proteins can in turn lead to tailored biological outputs (e.g., gene activation). The chemical inducer (e.g., ABA) can be modified into a prodrug version that becomes activatable by a user-chosen signal such as, for example, a secondary messenger, a metal ion, light, or other endogenous signal in local cellular environments. Using a prodrug responsive to a unique signal, presented only in a solid tumor microenvironment, and combined with the CIP technology, tailored therapeutic outputs can be specifically and locally produced for selected tumors. When integrating prodrug/CIP technology with CAR T cell engineering, CAR T cells (e.g., next generation enhanced CAR T cells, the CAR Plus T cells) can be created that express desired therapeutic proteins (e.g., PD-L1 mAb, IL-12, or TRAIL) and achieve targeted tumor killing in solid tumor sites with limited collateral damage to healthy tissues.

Several proteases are overexpressed in solid tumor microenvironments, which degrade extracellular matrixes and facilitate cancer metastasis. For example, elevated levels of cathepsin B, MMP-2, MMP-9, uPA, plasmin, caspase 3, and others have been associated with solid tumors (e.g., melanoma) and their invasiveness. Many cancer prodrugs have been developed to be activatable by these proteases. These prodrugs typically have optimized protease-specific peptide substrates conjugated to cytotoxic small molecules that mask the activity of these molecules until these peptide “caging” groups are cleaved off in the presence of overexpressed proteases in cancer environments. Prodrugs for chemically-induced proximity (CIP) chemical inducers (e.g., ABA) can be designed to become active only inside solid tumor local environments. Locally-produced ABA, for example, can readily induce the association of corresponding ABI and PYL fusion proteins within seconds and induced desired biological effects even if any possible diffusion of ABA occurs that may dilute ABA to become under the active concentration.

While described herein in the context of an exemplary embodiments in which the CIP chemical inducer is ABA, the technology described herein may employ other CIP inducers. The ABA CIP system has been proven to be suitable for the proposed strategy and ABA has been evaluated by EPA for the safety in human consumption. However, prodrugs may be designed under the technology platform described herein based on other CIP inducers such as, for example, rapamycin or gibberellic acid (GA), FK506, auxin, or any synthesized molecule that can induce the association of two proteins. DNA plasmids made in this study can be rapidly modified for the use of these alternative CIP inducers. FIG. 7 shows an exemplary rapamycin-based prodrug. Rapamycin and its derivatives have either been approved by FDA as anti-cancer drugs (e.g., temsirolimus (TORISEL, Pfizer, Inc., New York, NY) and everolimus (AFINITOR, Novartis Pharmaceuticals Corp., Basel, Switzerland) or showed great promise for cancer treatment (e.g., RAPALINK-1, APExBIO Technology LLC, Houston, TX). When rapamycin is generated from corresponding prodrugs by proteases, it can not only induce the therapeutic effects from the CAR Plus T cells but itself can also act as an anticancer drug to kill cancer in local solid tumor environments.

CIP-mediated signal-triggered gene expression technology can be incorporated into CAR T cells to generate CAR Plus T cells (CAR+ T cells) that produce and secrete an immune checkpoint inhibitor (e.g., PD-L1 mAb) and/or other therapeutic proteins such as, for example, IL-12 (which enhances the activation of cytotoxic T cells and NK cells) and/or TRAIL (which induces apoptosis of cancer cells expressing death receptors). The production of these therapeutic proteins occurs only when CAR T cells located within solid tumor microenvironments are activated by local tumor-specific cues (e.g., overexpressed proteases, hypoxia, etc.) (FIG. 1). Such a “double targeting” strategy-i.e., recognizing both tumor surface antigens (through CARs) and tumor microenvironment reactivity properties (through ABA prodrug activation)-provides unparalleled therapeutic specificity. Locally generated PD-L1 mAb and IL-12 can enhance the antitumor activity and/or persistence of CAR Plus T cells in the solid tumor environment without causing collateral damage systemically. Combined with TRAIL production (or any other antitumor therapeutic agents), which selectively induces the apoptosis of cancer cells, these enhanced T cells can offer additional therapeutic benefits over current immunotherapy approaches.

FIG. 1 also illustrates an embodiment involving a split CAR that can be activated by a cancer signal through a caged inducer (e.g., ABA). In this embodiment, T cells contain an engineered split CAR that remains inactive and cannot activate the T cell unless the split CAR is reconstituted into complete form in the presence of CIP inducers and, at the same time, is engaged in tumor antigen binding. An inducer-based (e.g., ABA-based) prodrug can be used to integrate signal-triggered CIP technology with the split CAR strategy to generate the next generation tumor-activated CAR T cells (the CAR Plus T cells). The activation of these T cells and the production of immune checkpoint inhibitors only occur when CAR Plus T cells target to tumor sites, bind to the chosen tumor antigen, and are time exposed to uncaged ABA that can only be activated by the chosen cancer signal in tumor environment (i.e., overexpressed protease) (FIG. 1). Locally activated CAR Plus T cells and locally generated anticancer therapeutic proteins (e.g. PD-L1 mAb and TRAIL) may produce synergistic and enhanced tumor killing effects with minimal collateral damage of healthy tissues.

An exemplary embodiment using ABA as an inducer (CAR Plus T cell/ABA prodrug strategy) can be tested against, for example, melanoma as a model system. One can design an ABA prodrug that is activatable by, for example, a protease associated with the solid tumor microenvironment (e.g., cathepsin B or MMP-2/9) or hypoxia in the solid tumor microenvironment. Also, for testing in a melanoma model system, one can design a DNA construct encoding a split CAR that recognizes the melanoma antigen chondroitin sulfate proteoglycan 4 (CSPG4) and can be reconstituted by ABA, as well as ABA-inducible gene cassettes that produce secreted PD-L1 mAb. The engineered melanoma-inducible CAR Plus T cells can be evaluated in the melanoma model system to test their cancer-triggered killing effects.

The chemical-induced proximity (CIP) technology has been applied to engineer human and other mammalian cell lines to generate specified biological responses (e.g., gene activation, signal transduction, etc.) upon stimulation by various cellular or artificial signals (e.g., H₂O₂, Fe²⁺, and light). Many cancer prodrugs activatable by proteases and other cancer specific signals have been developed. These protease-responding prodrugs typically have a short peptide that is an optimized substrate for a particular cancer-associated protease. Since there are many cancer-associated proteases, there are many possible protease-optimized peptides. One can select one of these short peptides to be conjugated to a cytotoxic small molecule through a linker cleavable by the protease. These optimized peptides can be conjugated onto ABA to generate cancer-responding ABA prodrugs.

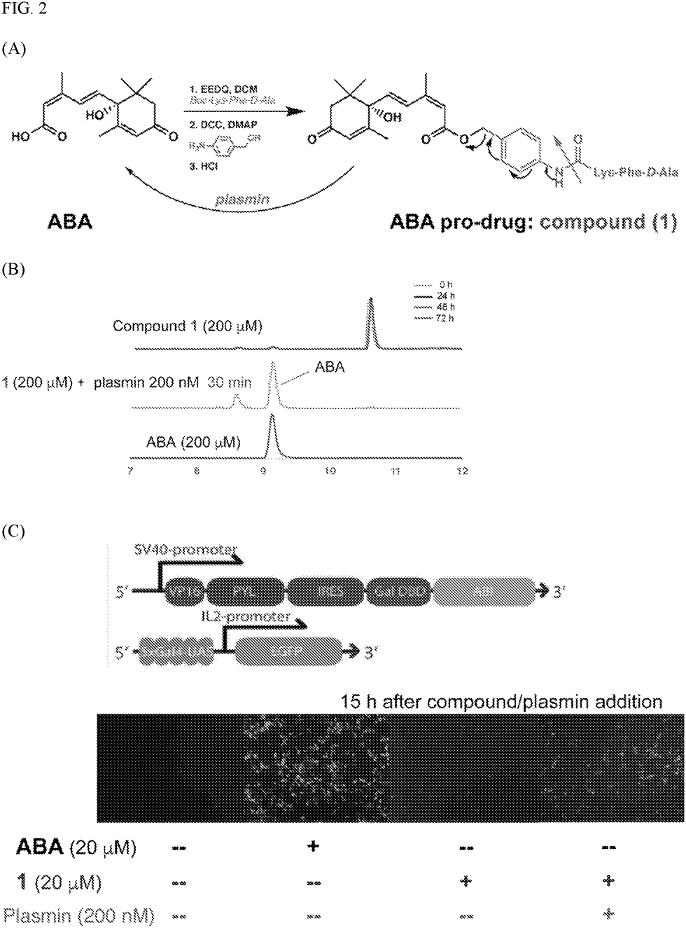

The chemical inducer can be designed to be responsive to hypoxia or the presence of a particular protease in the solid tumor microenvironment. For example, ABA prodrugs for melanoma-associated pericellular or extracellular proteases including cathepsin B, MMP-2/9, uPA, caspase 3, and plasmin (which can be converted from plasminogen by uPA) can be synthesized. Exemplary peptides identified as being suitable substrates for each protease (Table 1) can be conjugated at the C-terminus of the peptide via a linker to the chemical inducer (e.g., the carboxylate group of ABA) using, for example, the chemistry shown in FIG. 2A. In this way, a panel of prodrugs can be generated, each one designed to be cleaved by a targeted protease.

TABLE 1

Amino acid sequences of peptide substrates of exemplary proteases

| Protease | Peptide Substrate Sequences | SEQ ID NO |
|---|---|---|
| Cathepsin B | Val-Ala | 1 |
| | Gly-Phe-Leu-Gly | 2 |
| | D-Ala-Phe-Lys-Lys | 3 |
| Plasmin | D-Ala-Phe-Lys | 4 |
| | D-Val-Leu-Lys | 5 |
| | D-Ala-Phe-Lys-Lys | 6 |
| uPA | D-Ala-Phe-Lys | 7 |
| | Gly-Gly-Gly-Arg-Arg | 8 |
| MMP-2/-9 | Gly-Ile-Leu-Gly-Val-Pro | 9 |
| | Gly-Pro-Leu-Gly-Ile-Ala-Gly-Gln | 10 |
| | Glu-Pro-Cit-Gly-Hof-Tyr-Leu | 11 |
| Caspase 3 | Asp-Glu-Val-Asp-Pro | 12 |
| | Lys-Gly-Ser-Gly-Asp-Val-Glu-Gly | 13 |

The chemical stability of the prodrug compounds can be examined by incubating them in, for example, TRIS buffer at 37° C. for 24 hours without adding enzyme that cleaves the prodrug caging group. The prodrug compounds can be incubated with different amounts of corresponding recombinant or purified enzyme for varied time periods at 37° C. HPLC analysis can be used to follow the conversion of prodrug into the active chemical inducer. Each prodrug also can be treated with multiple enzymes and followed by HPLC to examine the specificity of the compounds. In this way, one can identify the minimal time, selectivity, and/or protease concentration required to convert each prodrug into active chemical inducer.

One also can examine the activation of prodrugs in reporter cells. For example, the cellular stability and the efficiency to generate functional ABA from an ABA prodrug can be examined using the ABA-inducible EGFP reporter cells. Cells can be incubated with ABA prodrugs with or without the appropriate prodrug-cleaving enzyme for varied time periods. The production of EGFP can be analyzed using fluorescence microscopy and a fluorescence plate reader.

For example, a plasmin-responsive ABA prodrug was synthesized using standard peptide coupling chemistry (FIG. 2A). In this exemplary embodiment, the ABA prodrug is cleaved by plasmin at the C-terminal of the peptide unit to reveal a linker that can then undergo a self-cleavage process to generate free ABA (FIG. 2A). Similar synthesis procedures can be applied to make other peptide-conjugated ABA prodrugs. HPLC analysis was used to analyze chemical stability and plasmin-dependent conversion of the plasmin-responsive ABA prodrug into ABA. The plasmin-responsive ABA prodrug was incubated with or without plasmin in TRIS buffer (pH 7.4) at 37° C. for different time periods. After the quick removal of plasmin by size exclusion spin column, HPLC was used to quickly identify the product species. The plasmin-responsive ABA prodrug is chemically stable within the tested 48-hour period and can be fully converted to ABA within 30 minutes of exposure to plasmin (FIG. 2B). ABA generated from the ABA prodrug is biologically functional to activate gene expression. By incubating the ABA-inducible 293T EGFP reporter cell line (that produces green fluorescent protein EGFP in the presence of functional ABA; Zeng et al., 2015. ACS Chemical Biology 10:1404-1410) with the plasmin-responsive ABA prodrug in the presence or absence of plasmin, the cellular stability and the plasmin-induced production of ABA and EGFP expression in cell culture was established. The plasmin-responsive ABA prodrug responds to plasmin and induces EGFP expression (FIG. 2C).

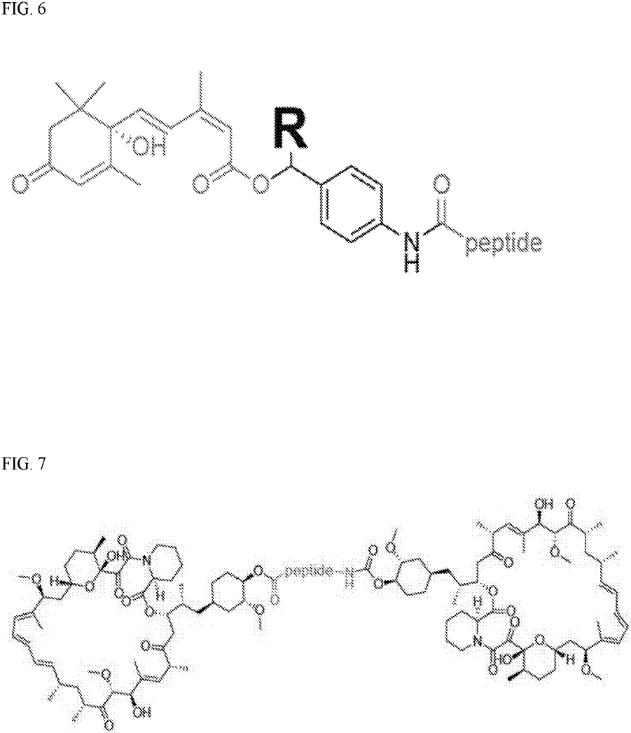

There is an ester linkage in the protease-responsive ABA prodrug that may be susceptible to esterase degradation in cells. To increase the stability of prodrugs in vivo, one can use modified versions of the prodrug that incorporates, for example, a bulky chemical substituent near the ester linkage (e.g., “R” in FIG. 6). Suitable bulky substituent groups include, for example, a tert-butyl or a phenyl group, which are known to increase the stability of ester bonds in similar compounds against esterases.

Because the chemical inducer (e.g., ABA) is linked to the protease substrate peptides at a distance through a linker, it is unlikely that the inducer will interfere with the effectiveness of the peptide cleavage by proteases. However, if in certain embodiments the inducer does interfere with the effectiveness of the peptide cleavage, one can replace amino acid sequences of the peptides or the linker to identify optimized structures of the prodrugs. One also can synthesize and test prodrug design for other melanoma-associated proteases such as, for example, FAP and KLK6).

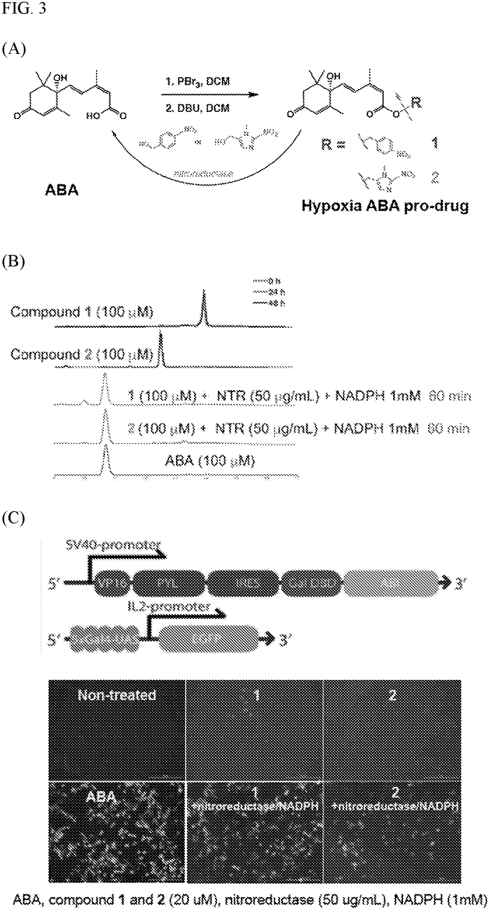

In another example, example, two hypoxia-responsive ABA prodrugs were synthesized using standard chemistry (FIG. 3A). In these exemplary embodiments, the hypoxia-responsive ABA prodrugs are cleaved by 1-electron reductase to generate free ABA (FIG. 3A). HPLC analysis was used to analyze chemical stability and hypoxia-dependent conversion of the hypoxia-responsive ABA prodrugs into ABA. The ABA prodrug was incubated with or without nitroreductase in TRIS buffer (pH 7.4) at 37° C. for different time periods. After the quick removal of nitroreductase by size exclusion spin column, HPLC was used to quickly identify the product species. The hypoxia-responsive ABA prodrugs are chemically stable within the tested 48-hour period and can be fully converted to ABA within 60 minutes of NADPH-dependent nitroreduction (FIG. 3B).

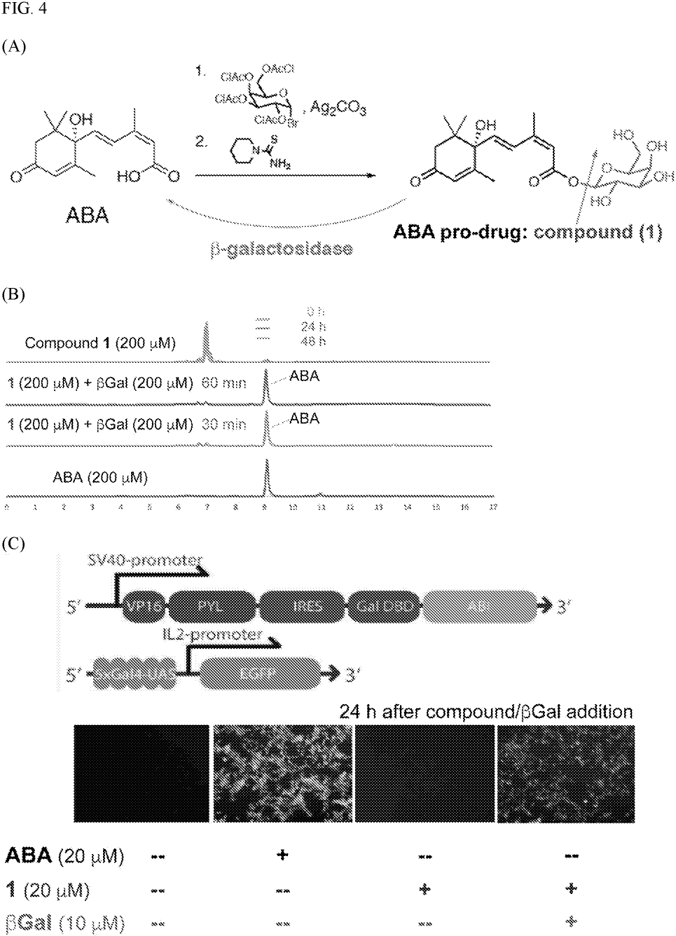

In another example, a β-galactosidase-responsive ABA prodrug was synthesized using standard saccharide coupling chemistry (FIG. 4A). In this exemplary embodiment, the galactose moiety is cleaved from the βGal-responsive ABA prodrug by β-galactosidase to generate free ABA (FIG. 4A). HPLC analysis was used to analyze chemical stability and βGal-dependent conversion of the β-galactosidase-responsive ABA prodrug into ABA. The βGal-responsive ABA prodrug was incubated with or without β-galactosidase in TRIS buffer (pH 7.4) at 37° C. for different time periods. After the quick removal of β-galactosidase by size exclusion spin column, HPLC was used to quickly identify the product species. The βGal-responsive ABA prodrug is chemically stable within the tested 48-hour period and can be fully converted to ABA within 30 minutes of exposure to β-galactosidase (FIG. 4B). ABA generated from the hypoxia-responsive ABA prodrug is biologically functional to activate gene expression. By incubating the ABA-inducible 293T EGFP reporter cell line (that produces green fluorescent protein EGFP in the presence of functional ABA; Zeng et al., 2015. ACS Chemical Biology 10:1404-1410) with the βGal-responsive ABA prodrug in the presence or absence of β-galactosidase, the cellular stability and the βGal-induced production of ABA and EGFP expression in cell culture was established. The βGal-responsive ABA prodrug can responds to β-galactosidase and induces EGFP expression (FIG. 4C).

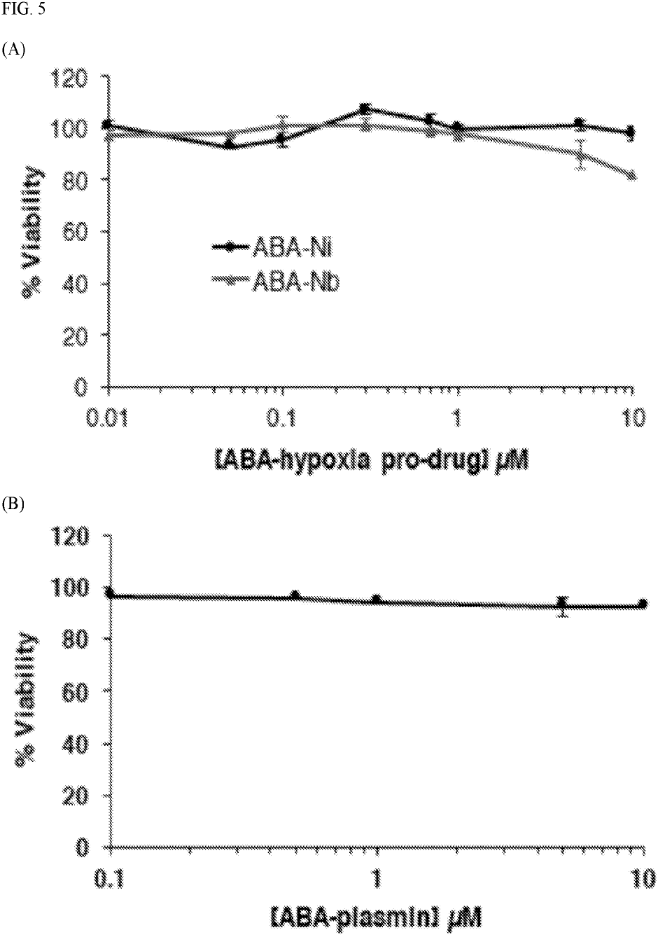

FIG. 5 provides data showing that the prodrugs alone are no cytotoxic. CHO cells were treated with varying concentrations of the two hypoxia-responsive ABA-prodrugs for 24 hours, then evaluated for cytotoxicity using an MTT assay. Cytotoxicity of the hypoxia-responsive ABA prodrugs versus a DMSO negative control is shown in FIG. 5A. Separately, CHO cells were treated similarly with the plasmin-responsive ABA prodrug. Cytotoxicity of the plasmin-responsive prodrug versus a DMSA negative control is shown in FIG. 5B.

This disclosure also describes DNA constructs that encode therapeutic polypeptides and are inducible by the chemical inducer through the CIP platform. The constructs generally include one or more exogenous polynucleotides that encode polypeptide components of the CIP platform and/or therapeutic polypeptides. The exogenous polynucleotides may be introduced into a T cell individually. Alternatively, two or more exogenous polynucleotides may be provided in a single vector.

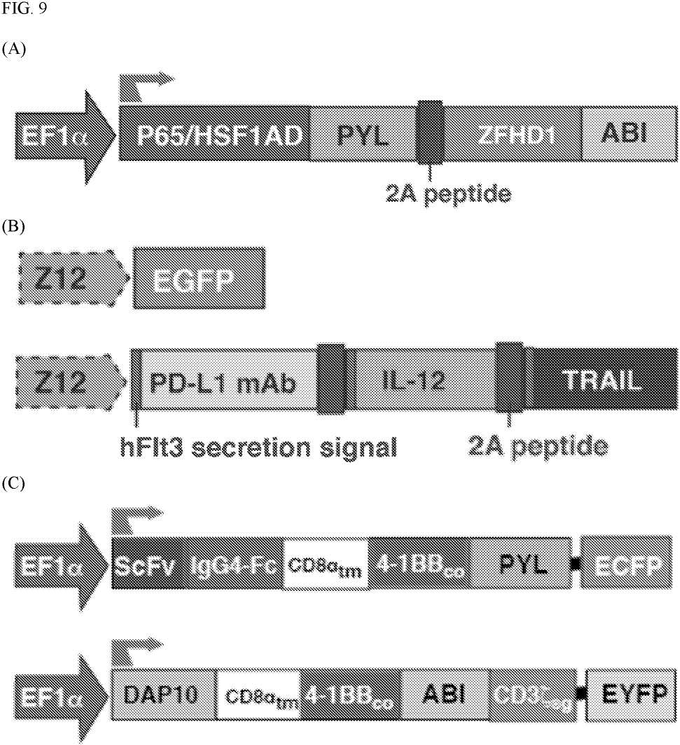

FIG. 9 illustrates various exemplary constructs in which one or more coding regions are operably linked to a regulatory sequence that controls expression of the coding regions. The plasmids encoding the ABA inducible system are designed to include cloning sites that allow simple and standard operations to insert any coding region encoding a protein, polypeptide, or peptide fragment that may be desired for a specified application. All of the cloned cassettes can be readily transferred into retroviral DNA vectors for retrovirus production. As used herein, “coding region” refers to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Regulatory sequences include, for example, promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

One exemplary set of constructs encodes ABA-inducible cassettes including ABA-dimerizable split transcriptional activator (P65/HSF1AD-PYL and ZFHD1-ABI linked by a self-cleaving 2A peptide; Szymczak et al., 2004. Nature biotechnol 22:589-594) and inducible secreted therapeutic proteins upon ABA stimulation (FIG. 9A). The ZFHD1 DNA binding domain (Pomerantz et al., 1995. Science 267:93-96) and the P65 (aa 361 - 551)/HSF1 (aa 406 -529) hybrid transcriptional activation domain (Pollock et al., 2000. Proc Nat Acad Sci USA 97:13221-13226) are selected for this exemplary construct because they are from human proteins and have been used for gene therapy applications.

Exemplary therapeutic proteins that can be expressed by the CAR T cells include FLAG tagged PD-L1 mAb, IL-12, and/or TRAIL (FIG. 9B). An exemplary inducible construct can contain a Z12 sequence (which contain 12 copies of the ZFHD1 consensus recognition sequence; Pomerantz et al., 1995. Science 267:93-96) upstream of the DNA sequences encoding the therapeutic proteins. These therapeutic proteins can be expressed as a polypeptide with each protein linked by a 2A peptide that subsequently undergoes auto-processing to give individual proteins. Alternatively, however, one or more therapeutic proteins may be expressed separately from other therapeutic proteins. The therapeutic proteins can include the N-terminal human Flt3 signal sequence to facilitate secretion out of T cells.

Another set of constructs can be made based on a split CAR targeting CSPG4 (FIG. 9C). The split CAR that has two components. One component encodes a CSPG4-recognizing scFv (Wang et al., 2011. Cancer Res 71:7410-7422) with IgG4-Fc linked to human CD8 transmembrane domain, 4-1BB costimulatory domain (Zhong et al., 2010. Mol Ther 18:413-420), PYL, and ECFP. The other component encodes an ectodomain of DNAX-activating protein 10 (DAP10; Wu et al., 1999, Science 285:730-732) linked to CD8 transmembrane domain, 4-1BB costimulatory domain, ABI, CD3z immunoreceptor tyrosine-based activation motifs (ITAMs; Weiss et al., 1994, Cell 76:263-274) and EYFP (Wu et al., 2015, Science 350:aab4077). CSPG4 is expressed on melanoma cell surface that is associated with the malignant progression of melanoma. Fluorescent protein FRET pairs ECFP and EYFP on each half of the split CAR allows one to monitor dimerization by, for example, fluorescence microscopy.

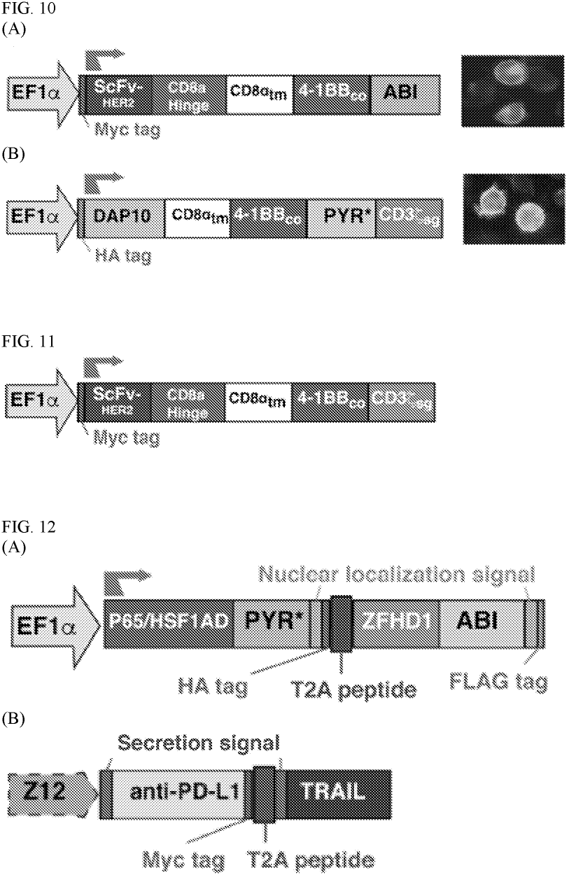

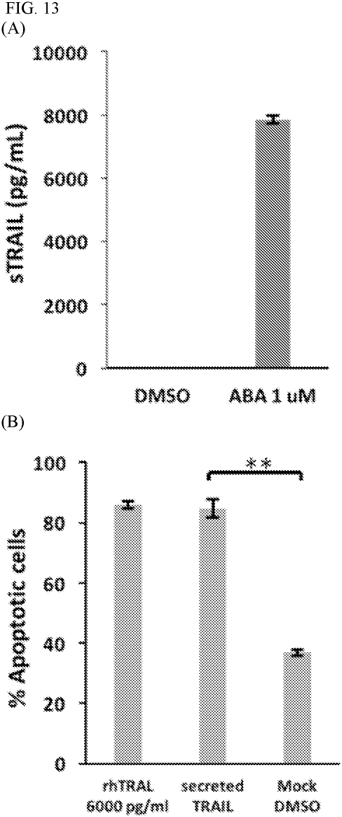

FIG. 11 shows a conventional CAR construct. FIG. 10 and FIG. 12 show split CAR constructs in which inducible gene expression depends on the presence of a small molecule dimerizer. FIG. 10A shows one half of an exemplary split CAR construct that includes a human HER2-recognizing scFv, the CD8 hinge region fused to CD8 transmembrane domain (CD8tm), 4-1BB costimulatory domain, and ABI. FIG. 10B shows the second half of the split CAR construct, which includes the ectodomain of DNAX-activating protein 10 (DAP 10) linked to CD8tm, 4-1BB, PYR*(F61L/A160C), and CD3ζ immunoreceptor tyrosine-based activation motifs (ITAMs). In another exemplary embodiment, FIG. 12A shows one half of an exemplary split CAR construct that includes an ABA-inducible gene cassette includes an ABA-dimerizable nuclear localized (by nuclear localization sequence, NLS) split transcriptional activator (P65/HSF1AD-PYR*-NLS and ZFHD1-ABI-NLS linked by a self-cleaving T2A peptide in the encoding plasmid. FIG. 12B shows the second half of the split CAR construct, which includes Z12-driven inducible therapeutic genes (expressing anti-PD-L1 and TRAIL linked by T2A in plasmid. ZFHD1 DNA binding domain (DBD) and P65 (aa 361-551)/HSF1 (aa 406-529) hybrid transcriptional activation domain are previously developed for gene therapy applications. The Z12 sequence contains 12 copies of consensus sequence recognized by ZFHD1-DBD.

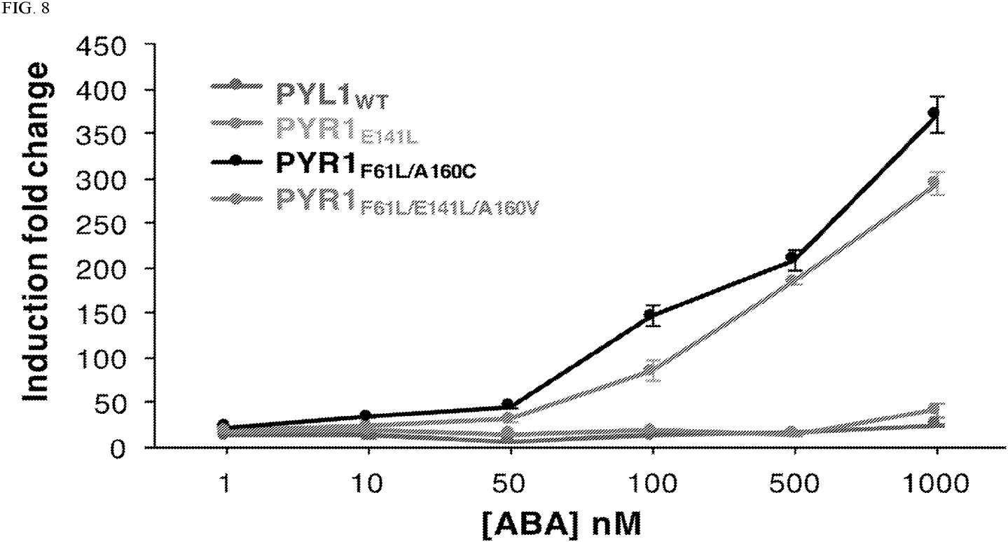

The constructs in FIG. 10 and FIG. 12 employ an engineered ABA-inducible CIP protein pair in which PYL1 (see, e.g., the constructs of FIG. 9) is replaced with different PYR1 mutants, including E141L, F61L/A160C, and F61L/E141L/A160V mutations. Using an ABA-inducible luciferase expression assay, PYR1F61L/A160C and PYR1E141L were identified as offering significantly enhanced ABA potency, thereby and allowing ABA doses at as low as 50 nM to 100 nM to achieve satisfactory induction (FIG. 8). The increased ABA potency may be beneficial for constructs intended for in vivo applications.

DNA plasmids made as described immediately above can be transfected into wild type HEK293T cells and be treated with (or without) ABA to induce the production of secreted PD-L1 mAb, IL-12, and TRAIL, or EGFP. To confirm the ABA-induced expression of PD-L1 mAb, IL-12, and TRAIL, at different time periods after ABA addition (e.g., four hours to 48 hours), cell culture supernatants can be analyzed by standard commercial ELISA kits (for IL-12 and TRAIL) or by immunoprecipitation using anti-FLAG antibody followed by western blotting (for PD-L1 mAb). The expression of EGFP and mCherry can be examined by fluorescence microscopy.

This disclosure further describes cells modified to include one or more of the constructs described immediately above. For example, a retrovirus constructed to include the one or more of the polynucleotide constructs may be introduced into human Jurkat T cells (Schneider et al., 1977. Internat J Cancer 19:621-626). Jurkat T cells can be transduced, for example, twice with these retroviruses on day 1 and day 3. On day 5, inducer (e.g., 20 µM ABA) can be added to the cells to induce the expression of proteins encoded by the construct (e.g., EGFP or PD-L1 mAb/IL-12/ TRAIL). The Jurkat T cells can be assayed to confirm the expression of inducible protein (e.g., EGFP) and mCherry from, for example, day 6 to day 9 to evaluate the transduction efficiency using, for example, flow cytometry analysis. The supernatant of cell culture media can be analyzed by ELISA (for. e.g., IL-12 and TRAIL) or by immunoprecipitation/western blotting (for, e.g., PD-L1 mAb) to confirm the induced production of secreted proteins from the CAR Plus T cells.

The modified cells may be used to investigate the response of CAR Plus T cells to CIP inducer prodrugs when co-cultured with solid tumor cells such as, for example, melanoma cells. To study the efficiency of CIP inducer prodrugs in respond to endogenous levels of extracellular proteases produced by solid tumor cells, the CAR Plus Jurkat T cells generated as described above can be co-cultured with different solid tumor cell lines (e.g., melanoma cell lines A375, B16, SK-MEL-2, SK-MEL-24, MV3, BLM and A2085) that are known to overexpress extracellular proteases that can be targeted (e.g., cathepsin B, MMP2/9, uPA, and/or caspase 3). The cells can be treated with CIP inducer prodrugs at varied concentration (10 µM to 50 µM). Flow cytometry analysis can be used to follow the expression of EGFP at different time points (24 hours to 72 hours) after the addition of prodrug. The cell culture supernatant can be examined by ELISA or immunoprecipitation/western blotting to monitor the production of, for example, secreted PD-L1 mAb, IL-12, TRAIL, or other therapeutic polypeptide.

The constructs described above also can be used to can be used to transduce primary human T cells. Purified primary human T cells—e.g., isolated from peripheral blood-can be activated by OKT3 (an anti-human CD3 antibody) and transduced by, for example, a retrovirus. The resulting CAR Plus T cells can then be co-cultured with solid tumor cells (e.g., A375, B 16, SK-MEL-2, SK-MEL-24, MV3, BLM, or A2085) and treated with a CIP inducer prodrug for different time periods (e.g., 24 hours to 72 hours). ELISA or immunoprecipitation/Western blotting can be used to monitor the production of desired proteins in the cell culture supernatant. Flow cytometry analysis can be used to monitor the expression of EGFP and mCherry.

After co-culturing the modified T cells with solid tumor cells (e.g., melanoma cells) and treating these cells with the CIP inducer (e.g., ABA) prodrug, one can examine the targeting of CAR Plus T cells to the solid tumor cells bearing CSPG4 by observing the mCherry-expressing red fluorescent T cells (non-adherent cells) binding to adherent solid tumor cells using fluorescence microscopy. To assess the enhanced ability of the modified T cells to kill the solid tumor cells, one can compare cell killing of the CIP inducer (e.g., ABA) prodrug/CAR Plus T cell system described herein versus standard CAR T cells or non-CAR T cells. Solid tumor cells (e.g., melanoma cells) and the T cells can be co-cultured under one of the following conditions: (i) with CAR Plus T cells and CIP inducer (e.g., ABA) prodrug; (ii) With CAR Plus T cells but no ABA prodrugs; (iii) with standard CAR T cells only (no prodrug); and (iv) with non-transduced T cells only. Under conditions (i), PD-L1 mAb, IL-12 and TRAIL are produced, thereby enhancing T cell activity (by PD-L1 mAb and IL-12) and providing extra tumor cell cell killing power (by TRAIL). Under conditions (ii), no therapeutic protein is produced and thus, the modified T cells provide a similar effect as standard CAR T cells.

While described above in the context of an exemplary embodiments in which the solid tumor is a melanoma tumor, the constructs and methods described above can involve constructs designed to target other solid tumors such as, for example, a colon cancer tumor, a prostate cancer tumor, a breast cancer tumor, a lung cancer tumor, a skin cancer tumor, a liver cancer tumor, a bone cancer tumor, an ovarian cancer tumor, a pancreatic cancer tumor, a kidney cancer tumor, a brain cancer tumor, or a head and neck cancer tumor. In some cases, the constructs and methods described herein also can involve constructs designed to target liquid tumors such as, for example, lymphoma.

Thus, while described above in terms of an exemplary embodiment in which therapeutic proteins are proteins selected for treating melanoma solid tumors—e.g., PD-L1 mAb, IL-12, and TRAIL—the constructs and methods described above can involve constructs designed to express alternative therapeutic polypeptides. Alternative therapeutic polypeptides include, for example, an immune checkpoint inhibitor, a cytokine, a cancer-specific therapeutic protein, a protein or polypeptide that activates T cells, a protein or polypeptide that enhances T cell persistence, and/or a protein or polypeptide that has anti-tumor activity. Exemplary immune checkpoint inhibitors include a monoclonal antibody or fragment thereof (e.g., an scFv) that specifically binds to PD-1, PD-L1, CTLA4, 4-1BB (CD137), and/or OX40. Exemplary cytokines include IFNα, IFNβ, INFγ, IL-2, IL-7, IL-15, IL-18, and/or IL-21. Cancer-specific therapeutic proteins include thrombospondin 1 (TSP1), PEX, a magainin, a cecropin, a defensin, pleurocidin, and/or a Bax-derived pore-forming peptide.

The scFv unit of CAR can be tailored to the particular solid tumor being treated. Thus, for example, the scFv unit of the CAR can include an scFv that specifically binds to an alternative melanoma antigen (e.g., GD2 or CD20) or other cancer associate antigen. Exemplary alternative cancer-associated antigens, with exemplary targeted tumor listed in parentheses, include α-folate receptor (e.g., ovarian cancer and epithelial cancers), CAIX (e.g., renal cell carcinoma), CD19 (e.g., B cell malignancies, ALL, CLL, lymphoma), CD22 (e.g., B cell malignancies), CD23 (e.g., CLL), CD 24 (e.g., pancreatic adenocarcinoma), CD30 (e.g., lymphomas and Hodgkin lymphoma), CD33 (e.g., AML), CD38 (e.g., Non-Hodgkin lymphoma), CD44v⅞ (e.g., cervical carcinoma), CEA (e.g., colorectal cancer), EGFRvIII (e.g., glioblastoma), EGP-2 (e.g., multiple malignancies), EGP-40 (e.g., colorectal cancer), EphA2 (e.g., glioblastoma), erb-B2 (e.g., breast cancer, prostate cancer, colon cancer, and other tumors), erb-B 2,3,4 (e.g., breast cancer), FBP (e.g., ovarian cancer), fetal acetylcholine e receptor (e.g., rhabdomyosarcoma), G_{D2} (e.g., neuroblastoma, melanoma, Ewing sarcoma), G_{D3} (e.g., melanoma), Her-2 (e.g., medulloblastoma, pancreatic adenocarcinoma, glioblastoma, osteosarcoma, ovarian cancer, breast cancer), HMW-MAA (e.g., melanoma), IL-11Rα (e.g., osteosarcoma), IL-13R-α2 (e.g., glioma, glioblastoma, medulloblastoma), KDR (e.g., tumor neovasculature), κ-light chain (e.g., B cell malignancies), Lewis Y (e.g., various carcinomas, epithelial-derived tumors), L1-cell adhesion molecule (e.g., neuroblastoma), MAGE-A1 (e.g., melanoma), mesothelin (e.g., mesothelioma), MUC-1 (e.g., breast cancer, ovarian cancer), MUC16 (e.g., ovarian cancer), NKG2D ligands (myeloma, ovarian cancer, other tumors), NY-ESO-1 (157-165) (e.g., multiple myelomas, oncofetal antigen (h5T4) (e.g., various tumors), PSCA (e.g., prostate carcinoma), PSMA (e.g., prostate cancer, tumor vasculature), ROR1 (e.g., B-CLL, mantle cell lymphoma), TAG-72 (e.g., adenocarcinoma), and/or VEGF-R2 (tumor vasculature).

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Cell Lines

Human embryonic kidney cell (HEK 293T), EGFP-expressing human embryonic kidney (HEK 293-GFP), Chinese Hamster Ovary (CHO) and HeLa cells were maintained in our laboratory. Human breast adenocarcinoma MDA-MB-231 were given by Dr. Lina Cui’s group (Chemistry Department, University of New Mexico, Albuquerque, NM). Cells were grown at 37° C., 5% CO₂, in Dulbecco’s modified Eagle’s medium (DMEM) (Gibco, Thermo Fisher Scientific, Inc., Waltham, MA) supplemented with 10% Fetal Bovine Serum (Omega Scientific, Inc., Tarzana, CA), 5% GLUTAMAX (Life Technologies, Therma Fisher Scientific, Inc., Carlsbad, CA) and 5% penicillin/streptomycin (Life Technologies, Therma Fisher Scientific, Inc., Carlsbad, CA).

### Plasmid Construction – Split CAR

The ABA-based split CAR system was constructed and modified from a previously reported system (Wu et al., Science 2015, 350(6258):aab4077). For the antigen recognition part, an scFv was cloned to target Her2, CSPG4, or CA-IX antigens. Lentiviral constructs were generated using second-generation lentiviral packaging plasmid as previously described (Hill et al., Nat Chem Biol 2018, 14(2):112-117).

### Plasmid Construction – Inducible Therapeutic Proteins

Construction of the sv-VPiGA, 5×IG and 5×I-Luciferase have been described previously (Liang et al., Sci Signal 2011, 4(164):rs2). Plasmid vectors expressing therapeutic proteins were made by replacing EGFP derived from 5IG with nucleotide sequences encoding for different therapeutic proteins: full length TRAIL (fl.TRAIL), secretable TRAIL ( sTRAIL), anti-CTLA4 scfv, anti-PDL1 scfv, and IL-12.

The pSV40-p65-PYL-2A-ZFHD1-ABI was derived from sv-VPiGA by replacing vp16 with the activation domain from the p65 subunit of human NF-κB, and by replacing ires-GAL4DBD with self-cleaving 2A peptide fused to the DNA-binding domain ZFHD1. The ABA-inducible therapeutic protein construct was made by inserting therapeutic genes (e.g. TRAIL, anti-PD-L1 scfv, IL-12) under control of a minimal IL-2 gene promoter and 12 ZFHD1 binding sites (Z12-IL2-therapeutic genes).

All plasmid constructs were purified using QIAprep spin miniprep purification kit (Qiagen, Hilden, Germany).

### Synthesis of ABA Prodrugs

Cancer-responding ABA prodrugs were synthesized by utilizing standard coupling chemistry as previously reported (Zeng et al., ACS Synth Biol 2017, 6(6):921-927). Different chemical moieties that are optimized substrates for specific cancer-associated proteases (e.g., peptide sequence for ABA-plasmin, Galactose for ABA-Gal, and nitrobenzyl/nitroimidazole for ABA-hypoxia) were conjugated at the C-terminal to the carboxylic acid group of ABA via cleavable linkers. Bulky substituents (e.g. “R” as tert-butyl or phenyl group in FIG. 6) were also introduced to improve the stability of the ester bond, if needed.

### Chemical Stability and Reactivity of ABA Prodrug Towards Chosen Signals

Appropriate volumes of stock ABA prodrugs in DMSO were diluted to different molar ratios for in vitro and cell culture assays. The in vitro stability test was done by incubating ABA prodrug (in DMSO) in Tris buffer of 0.1 M final concentration or PBS buffer (pH 7.4) up to 72 hours at 37° C. HPLC data were obtained at 0 minutes, 24 hours, 48 hours, and 72 hours. The chromatograms were collected by liquid chromatography (Dionex-UltiMate 3000 LC System with Acclaim 120 Å, C18, 3 µm analytical (4.6 × 100 mm), Thermo Fisher Scientific, Inc., Waltham, MA).

Assays to measure reactivity of ABA prodrugs towards corresponding signals were performed as follows:

For ABA-plasmin, various concentrations of plasmin were mixed with 200 µM ABA-plasmin prodrug, then incubated at 37° C. for 30 minutes. The mixture was then allowed to pass through 10 K centrifugal filters (VWR) at 15,000 rpm to remove the protease, and the filtered suspension was then quickly injected into HPLC and analyzed.

For ABA-Gal, various concentrations of beta-galactosidase were mixed with 200 µM ABA-Gal prodrug, then incubated at 37° C. for 30 minutes. The mixture was then allowed to pass through 10 K centrifugal filters (VWR) at 15,000 rpm to remove the protease, and the filtered suspension was then quickly injected into HPLC and analyzed.

For ABA-hypoxia, 100 µM of ABA-Ni or ABA-Nb was incubated with nitro-reductase enzyme (50 µg/mL) and NADPH (1 mM) at 37° C. for 60 minutes, followed by HPLC analysis.

All HPLC analyses were run at room temperature and monitored at 260 nm. A gradient of acetonitrile in 0.1% TFA and water containing 0.1 %TFA were used at a flow rate of 0.750 mL/min. Acetonitrile was increased from 5% to 75% at 15 minutes and kept constant for three minutes.

### EGFP Expression Experiments

HEK 293T-EGFP reporter cell lines were seeded in a 24-well plate at 10⁵ cells/well 24 hours prior to drug treatment. Appropriate volumes of ABA (as a control), ABA prodrugs, or ABA prodrugs with and without corresponding cancer-associated signals were added to the wells and the cells were incubated at 37° C. in a CO₂ incubator. Fluorescence images were taken at different time points following drug incubation.

### Apoptosis Assay and Quantification of Secreted TRAIL

For sTRAIL, apoptotic assays, 293T cells were transfected to express the new potent ABA-responsive split transcriptional activator and 5×Gal4 response elements controlling the expression of sTRAIL. Cell culture supernatant containing sTRAIL was collected after 24 hours treated with either ABA 1 µM or mock DMSO and incubated with TRAIL-sensitive MDA-MD-231 cancer cell lines for another 24 hours. Cell viability and apoptosis were assessed using a dead cell apoptosis kit for flow cytometry according to the manufacturer’s protocol (Invitrogen, Thermo Fisher Scientific, Inc., Carlsbad, CA). Briefly, cancer cells were harvested by trypsinization at 24 hours after drug treatment and were washed twice with cold PBS. Then 10⁶ cells, suspended in Annexin V binding buffer, were incubated with propidium iodide (1 µg/ml) and FITC-Annexin V (1:20 volume ratio) for 15 minutes at room temperature in the dark. Following which, 400 µl annexin-binding buffer was added and the stained cells were immediately analyzed by flow cytometry (ACCURI C6 cytometer, BD Biosciences, San Jose, CA). The concentration of secreted TRAIL, in the supernatant was determined via human TRAIL ELISA kit (PICOKINE, Boster Biological Technology, Pleasanton, CA).

### Cellular Toxicity Evaluation

Cytotoxicity of prodrugs were evaluated using MTT assay (MTT = 3-(4, 5-dimethylthiazol-2-yl)-2, 5-diphenyltetrazolium bromide). 10⁴ Hela cells/well were seeded in triplicate in a 96-well flat bottom plate and allowed to adhere for 24 hours at 37° C. in a CO₂ incubator. Cells were transfected with ABA-inducible EFGP reporter DNA plasmids. 24 hours post-transfection, cells were treated with various concentrations of the desired prodrug compounds. After 24 hours of incubation, culture medium was replaced with a fresh medium. Subsequently, 10 µL of MTT working solution (5 mg/mL in PBS) was added to each well and the plate was incubated for four hours at 37° C. The medium was then aspirated, and the formed formazan crystals were solubilized by adding 50 µL of DMSO per well for 30 minutes at 37° C. Finally, the intensity of the dissolved formazan crystal (purple color) was quantified at 540 nm using plate reader (SPECTRAMAX i3X, Molecular Devices LLC, San Jose, CA).

### Luciferase Reporter Assay

CHO cells (10⁴) were seeded in triplicate in a 96-well plate and allowed to adhere for 24 hours at 37° C. in a CO₂ incubator. Cells were transfected with ABA-responsive split transcriptional activator with PYL1 being replaced with PYR1 mutants including E141L, F61L/A160C, and F61L/E141L/A160V mutations, and 5×Gal4 response elements controlling the expression of firefly luciferase. 24 hours post-transfection, cells were incubated with varying concentration of ABA (or DMSO alone). Luciferase activity was detected using Luciferase Assay System, for luminometer according to manufacturer’s protocol (GLOMAX-MULTI detection system, Promega, Madison, WI). The triplicate data obtained for each mutant condition were averaged and normalized to DMSO treatment.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A T cell comprising:
a first exogenous polynucleotide that encodes a therapeutic polypeptide operably linked to a regulatory region inducible by inducer;
a second exogenous polynucleotide that encodes polypeptide components of a chemical induced proximity (CIP) complex; and
a third exogenous polynucleotide that encodes a chimeric antigen receptor that specifically binds to an antigen.

2. The T cell of claim 1 wherein the regulatory region binds components of a chemical induced proximity (CIP) expression system.

3. The T cell of claim 1 wherein presence of the inducer in the T cell induces assembly of the CIP expression system and expression of the therapeutic polypeptide.

4. The T cell of claim 1 wherein the inducer comprises abscisic acid (ABA), gibberellic acid (GA), or rapamycin.

5. The T cell of claim 1 wherein the chimeric antigen receptor comprises a split chimeric antigen receptor.

6. A polynucleotide comprising:
a coding region that encodes a therapeutic polypeptide operably linked to a regulatory region inducible by a chemical induced proximity (CIP) complex;
a plurality of coding regions encoding polypeptide components of the CIP complex.

7. A method of treating a solid tumor in a subject, the method comprising:
introducing into T cells exogenous polynucleotides to produce a modified T cell, the exogenous polynucleotides comprising:
a first exogenous polynucleotide that encodes a therapeutic polypeptide operably linked to a regulatory region inducible by inducer;
a second exogenous polynucleotide that encodes a chemical induced proximity (CIP) complex that is induced by an inducer molecule; and
a third exogenous polynucleotide that encodes an inducible split chimeric antigen receptor that specifically binds to an antigen expressed by cells of the solid tumor;
administering the modified T cell to the subject;
administering to the subject an inactive prodrug of the inducer, the inactive prodrug being activatable to the inducer by an enzyme secreted by cells of the solid tumor or an environmental condition of the solid tumor;
allowing the inactive prodrug to be converted to the active inducer; and
allowing the active inducer to enter the modified T cell and induce the CIP complex to express:
the therapeutic polypeptide; and
the chimeric antigen receptor.

8. The method of claim 7 wherein the T cells are obtained from the subject.

9. The method of claim 7 wherein the chimeric antigen receptor comprises a CIP-controlled split chimeric antigen receptor.

10. The method of claim 7, wherein the environmental condition of the solid tumor comprises hypoxia.
